(19) United States
(12) Patent Application Publication
(10) Pub. No.: US 2014/0165263 A1
(43) Pub. Date: Jun. 19, 2014

(54) FLUID REPELLENT ELASTOMERIC BARRIER

(71) Applicant: Ansell Limited, Richmond (AU)

(72) Inventors: Thi Hao Pham, Shah Alam (MY); Norman W. Keane, Sungai Buyloh (MY); Albert Khor, Shah Alam (MY); David M. Lucas, Kuala Lumpur (MY); Mohamad Izwan Jaafar, Shah Alam (MY); Khadzrul Ariff Mohamad Baki, Shah Alam (MY)

(21) Appl. No.: 14/107,420

(22) Filed: Dec. 16, 2013

Related U.S. Application Data
(60) Provisional application No. 61/738,660, filed on Dec. 18, 2012.

(51) Int. Cl. A61B 19/04; B05D 3/02; B05D 1/18

(52) U.S. Cl. CPC A61B 19/04; B05D 1/18; B05D 3/0254
USPC 2/164; 2/168; 427/2.3

(57) ABSTRACT

Fluid repellent coatings including a silicone coating for elastomeric gloves, and methods for treating gloves having elastomeric coatings, are disclosed.

### CROSS-REFERENCE TO RELATED APPLICATIONS

This application claims the benefit of priority to U.S. Application Ser. No. 61/738,660, filed Dec. 18, 2012, which is herein incorporated by reference in its entirety.

### BACKGROUND

1. Field of the Invention

Embodiments of the present invention generally relate to barriers and, more particularly, to elastomeric gloves or fabric gloves having elastomeric coatings comprising fluid repellent surfaces.

2. Description of Related Art

Protective gloves often comprise elastomeric materials, are thin and flexible, and can be used to help handle dangerous liquids safely and are used in many industries, such as in chemical laboratories, warehouses, and manufacturing plants as well as for medical procedures, such as surgeries. Fluid adhesion to gloves having elastomeric coatings reduces gripping properties and impedes visibility. Fluid repellency is therefore a desirable characteristic for gloves. For surgical gloves, blood repellency is an important characteristic as it provides the surgeon with a clearer view of their fingers and operating field during surgery, thereby enhancing the accuracy of the procedure. However, surgical procedures can last for several hours and, generally, the user finds it inconvenient or impractical to replace the gloves each time they contact and/or become covered with a fluid.

Therefore, a need exists for an elastomeric barrier having fluid repellent properties.

### SUMMARY

Embodiments of the invention comprise fluid repellent treatments for elastomeric materials and methods for treating elastomeric materials, substantially as shown in and/or described in connection with at least one of the figures herein, are disclosed as set forth more completely in the claims. Various advantages and features of the present invention will be more fully understood from the following description and drawings.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1A shows a visual water repellency test for polyisoprene (PI) gloves made more repellent with a conventional fluorocarbon treatment, or with a treatment according to embodiments of the present invention;

FIGS. 1B and 1C show a visual blood repellency test for PI gloves made more repellent with a conventional fluorocarbon treatment, or with a treatment according to embodiments of the present invention, the gloves shown immediately after dipping in blood, and 5 minutes thereafter, respectively;

FIG. 2A shows a visual water repellency test for PI gloves made more repellent with different amounts of the treatment of the invention;

FIGS. 2B and 2C show a visual blood repellency test for PI gloves made more repellent with different amounts of a treatment according to embodiments of the present invention, the gloves shown immediately after dipping in blood, and 5 minutes thereafter, respectively;

FIGS. 3A, 3B, and 3C show a visual water repellency test for PI gloves, natural rubber (NR) gloves, and polychloroprene (CR) gloves. The gloves treated according to embodiments of the invention are on the left sides of the figures, and the non-treated controls on the right;

FIG. 4 shows a visual blood repellency test for PI gloves made more repellent with embodiments according to the treatment of the invention (high dose), the gloves shown 5 minutes thereafter dipping in blood;

FIG. 5 shows a visual blood repellency test for NR gloves made more repellent with embodiments according to the treatment of the invention (high dose), the gloves shown 5 minutes thereafter dipping in blood;

FIG. 6 shows a visual blood repellency test for CR gloves made more repellent with embodiments according to the treatment of the invention (high dose), the gloves shown 5 minutes thereafter dipping in blood;

FIG. 7 shows the stress-strain curves for PI, NR, and CR gloves that were treated or untreated (control) with embodiments according to the treatment of the invention;

FIG. 8 shows the ultimate tensile strength (UTS) and elongation of break of PI, NR, and CR gloves that are sterilized, or sterilized and aged, measured (by %) relative to the treated glove without sterilization or aging;

FIG. 9 depicts a flow diagram for a method 900 to surface treat an article having an elastomeric surface, according to embodiments of the invention;

FIG. 10A shows a visual water repellency test for treated siliconized and treated non-siliconized PI gloves; and

FIGS. 10B and 10C show a visual blood repellency test for treated siliconized and treated non-siliconized PI gloves.

While the invention is described herein by way of example using several embodiments and illustrative drawings, those skilled in the art will recognize that the invention is not limited to the embodiments of drawing or drawings described. It should be understood that the drawings and detailed description thereto are not intended to limit the invention to the particular form disclosed, but on the contrary, the invention is to cover all modification, equivalents and alternatives falling within the spirit and scope of the present invention as defined by the appended claims.

The headings used herein are for organizational purposes only and are not meant to be used to limit the scope of the description or the claims. Also, as used throughout this application, the word “may” is used in a permissive sense (i.e., meaning having the potential to), rather than the mandatory sense (i.e., meaning must). Similarly, the words “include,” “including,” and “includes” mean including, but not limited to. The word “glove” means glove or glove liner.

### DETAILED DESCRIPTION

Embodiments of the present invention generally relate to fluid repellency treatments. For example, the fluid repellency treatments may be disposed on barriers such as fabrics, polymeric articles, such as polymeric gloves, or on supported gloves (fabric gloves having a polymeric coating disposed thereon). Embodiments according to the invention relate to such barriers comprising a combination of a first coating of hydrophobic chemical on the exterior surface with a second coating containing hydrophobic micro-particles to increase fluid repellency on the exterior surface and, optionally, a third coating comprising silicone.

The combination of coatings according to embodiments of the invention forms a barrier coating that is differentiated from prior repellent coatings based on conventional fluorine chemistry. Barriers coatings in accordance with the invention comprise a fluid repellency treatment providing a rough, 3D surface structure on which hydrophobic particles, for example, silica (silicon dioxide) micro-particles, are deposited onto a flexible substrate, thereby creating a super fluid repellent effect.

Embodiments according to the invention comprise a highly effective combination for increasing the fluid repellency of elastomeric barriers, which may be used with elastomeric polymers of the type used for forming flexible surgical gloves, such as natural rubber (NR), polychloroprene (CR), acrylonitrile butadiene copolymer (NBR) (such as carboxylated acrylonitrile butadiene copolymer), polyisoprene (PI), polyurethane (PU), styrene-butadiene, butyl rubber (copolymer of isobutylene with isoprene, or polymer of isobutylene), or combinations thereof.

Silicone coatings that can be used with embodiments according to the invention include a formulation comprising a dimethicone emulsion, a cationic emulsion of an amine-functional silicone polymer, and a nonionic polypropylene emulsion.

Microparticle dispersions that can be used with embodiments according to the invention include wherein the hydrophobic micro-particles can be those described in, for example, US Publ. No. 2010/0112204, US Publ. No. 2010/0159195, or U.S. Pat. No. 7,056,845, the entire disclosures of which are herein incorporated by reference in their entireties. The micro-particles of US Publ. No. 2010/0112204 are reacted with linking reagents, followed by reaction with hydrophobic groups that attach to the resultant linking groups. Hydrophobic entities include C₃-C₂₄ hydrocarbon or C2-C12 perfluorinated carbon backbones. The micro-particles may also comprise nanoparticles, so long as the ability to induce a lotus effect with water is retained. For example, the size range can be 0.01 to 10 micrometers. Other micro-particles include silica particles.

Hydrophobic chemicals for use with embodiments of the invention also include known commercial products, for example, Softgard M3 (soft chemicals, Italy), Oleophobol 7752 (Huntsman, Germany), Ruco-Gard AIR and Ruco-Dry DHY (Rudolf Chemie, Germany), Scotchgard® (3M Inc., Maplewood, Minn.), Zepel-B™ (Dupont, Wilmington, Del.), anionic perfluoropolyether based polyurethane and polytetrafluoroethylene (Fluorolink® 5049), and perfluoropolyether based triethosilane (Fluorolink® S10, available from Ausimont, Thorofare, N.J.), perfluoroalkyl acrylic co-polymer (such as Zonyl® 8300 available from Ciba Specialty, High Point, N.C.; and Scotchban™ FC-845 available from 3M, St. Paul, Minn.), perfluoroalkyl urethane (such as L-8977 available from 3M, St. Paul, Minn.), perfluoropolyether-modified polyurethane dispersion (such as Fluorolink™ P56 available from Ausimont, Thorofare, N.J.), fluorinated silicone polyester (such as Lambent™ WAX available from Lambent Technologies, Fernandina Beach, Fla.), polychlorotrifluoroethylene (such as Aclon™ PCTFE available from Honeywell, Morristown, N.J.), polyvinylidene fluoride dispersion (such as Unidyne™ TG available from Daikin America, New York, N.Y.), tetrafluoroethylene-hexafluoropropylene copolymer (such as Dyneon™ FEP available from 3M, Parsippany, N.J.), polyperfluoroethoxymethoxydifluoroethyl PEG phosphate (such as Fomblin™ HC/2-1000 available from Solvay Solexis, Houston, Tex.), Oleophobol® CP-SLA (an aqueous dispersion of perfluorinated acrylic copolymer), like hydrophobic chemicals, and combinations thereof.

A variety of fluorochemical, fluid repellent compounds suitable for use in accordance with embodiments of the present invention are known and are commercially available. One particular group of fluorochemical repellents are the polymers obtained by polymerizing an ethylenically unsaturated fluorochemical compound. The ethylenic unsaturation may be either in the alcohol or the acid portion of the ester molecule. Typically, the unsaturated radical in the alcohol portion of the ester may be the allyl radical or the vinyl radical. Typical unsaturated acids used to prepare the ester include acrylic acid, methacrylic acid and crotonic acid. In general, the perfluoro portion of the molecule is in the saturated portion of the molecule. The unsaturated portion of the molecule is typically not fluorinated in each instance. The acid and alcohols radicals may suitably contain from 2 to 6 carbon atoms excluding the carbonyl carbon of the acid. Examples of such monomers include vinyl perfluorobutyrate and perfluorobutyl acrylate. These monomers may be polymerized as homopolymers or as copolymers by normal emulsion polymerization techniques using free radical catalysts.

Examples of other suitable fluorochemical repellents for use in embodiments of the invention are those known and sold under the trademarks “Scotchgard® FC 208”, “Scotchgard® FC 210”, “Scotchgard® FC 232”, and Scotchgard® FC 319”, manufactured by the 3M Company, “Zepel™ B” manufactured by E. I. DuPont de Nemours and Co. and “Tinotop™ T-10” manufactured by Ciba-Geigy Ltd.

Of these materials “Scotchgard® FC 208” is an aqueous nonionic emulsion containing approximately 28% by weight of a modified fluorinated acrylic polymer: a substance believed to be of the following approximate general formula:

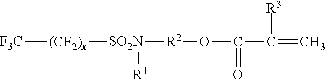

in which X is a value between 3 and 13 inclusive, R₁ is lower alkyl, such as methyl, ethyl, propyl, and the like, having 1-6 atoms R₂ is alkylene containing 1-12 carbon atoms and R₃ is H, methyl or ethyl. The product “Zepel™” is also available in emulsion form and while it is chemically different from the “Scotchgard®” products, it is a fluorochemical oil repellent containing fluorocarbon tails composed of CF₂ groups which may end in a terminal CF₃ group.

“Scotchgard® FC-319” is a solution of a compound similar to “FC-208” in an organic solvent. “Scotchgard® FC-232” is a dispersion of a fluorochemical resin in a mixture of water and methyl isobutyl ketone. “Zepel B™” is an aqueous cationic dispersion of a fluorochemical resin and is a product of E. I. Dupont de Nemours and Company. These products are believed to fall within the classes of compounds disclosed in the following patent specifications (compound descriptions incorporated herein by reference in their entirety): UK Pat. No. 971,732; Canadian Pat. No. 942,900; Canadian Pat. No. 697,656; French Pat. No. 1,568,181; French Pat. No. 1,562,070; German Pat. No. 1,419,505; U.S. Pat. No. 2,803,615; U.S. Pat. No. 2,826,564; U.S. Pat. No. 2,642,416; U.S. Pat. No. 2,839,513; U.S. Pat. No. 2,841,573; U.S. Pat. No. 3,484,281; U.S. Pat. No. 3,462,296; U.S. Pat. No. 3,636,085; U.S. Pat. No. 3,594,353; and U.S. Pat. No. 3,256,230.

Fluorolink® 5049 is a composition containing an anionic perfluoropolyether (PFPE) based polyurethane dispersion in water, polytetrafluoroethylene (PTFE) dispersion, isopropyl alcohol and methyl ethyl ketone, and is available from Solvay Solexis, Thorofare, N.J. Fluorolink® S10 is a composition containing a perfluoropolyether (PFPE)-based triethoxysilane dispersion in water, available from Solvay Solexis.

Wax dispersions for use as the hydrophobic chemical in accordance with embodiments of the invention, or as a supplement to a primary hydrophobic chemical (e.g., Freepel® 1225), include water-based wax dispersions such as, but are not limited to, synthetic wax (such as Freepel 11225 available from Noveon, Inc., Cleveland, Ohio); polyethylene wax (such as Michem™ ME available from Michelman, Cincinnati, Ohio; Luwax™ AF available from BASF, Parsippany, N.J.; Aquatec™ available from Eastman Chemical, Kingsport, Tenn.; and Jonwax™ available from S.C. Johnson Wax, Racine, Wis.); oxidized polyethylene wax (such as PoligenT WEI available from BASF, Parsippany, N.J.); ethylene acrylic acid copolymer EAA wax (such as Poligen™ WE available from BASF Parsippany, N.J.); ethylene vinylacetate copolymer wax (such as Aquacer™ available from BYK, Wallingford, Conn.); modified polypropylene wax (such as Aquaslip™ available from Lubrizol, Wickliffe, Ohio); silicone wax (such as DC 2503, DC2-1727, DC C-2-0563, DC 75SF and DC 580 available from Dow Corning, Midland, Mich.); Masilwax™ (available from Noveon, Cleveland, Ohio); Silcare™ 41M (available from Clariant, Charlotte, N.C.); fluoroethylene wax (such as Hydrocer™ available from Shamrock, Newark, N.J.); Carnauba wax (such as Slip-Ayd™ SL available from Daniel Products, Jersey City, N.J.); Fischer-Tropsch wax (such as Vestowax™ available from Degussa, Ridgefield, N.J.); and ester wax (such as Luwax™ E available from BASF, Parsippany, N.J.; and Lipowax™ available from Lipo, Paterson, N.J.), like waxes, and combinations thereof.

Optionally, fillers, resins, processing aids, cross-linkers, catalysts for cross-linking polymeric, elastomeric, or latex materials, such as natural rubber (NR), polychloroprene (CR), acrylonitrile butadiene copolymer (NBR) (such as carboxylated acrylonitrile butadiene copolymer), polyisoprene (PI), polyurethane (PU), styrene-butadiene, butyl rubber (copolymer of isobutylene with isoprene, or polymer of isobutylene), or combinations thereof and the like, as discussed above, can be used to further enhance the repellency and durability. These additional components can be incorporated within any elastomeric, polymeric, or latex compositions, which are then used with the hydrophobic chemical components of embodiments of the present invention to form a surface treated glove. Also, in certain embodiments, the elastomer for the unfoamed or foamed polymeric glove is predominantly NBR. In certain embodiments it is substantially (90% or more by weight) NBR.

In at least one embodiment of the invention, the polymeric glove may be formed of latex having commonly used stabilizers such as potassium hydroxide, ammonia, sulfonates, and the like, which may be incorporated within any composition described herein. And, in at least one embodiment, the latex may contain other commonly used ingredients such as surfactants, anti-microbial agents, fillers/additives and the like. For NBR formulations, acrylonitrile content can in certain embodiments be, for example, 28-34%, 35-37%, or 38-42%.

A fluid repellent coating, for example, a polymeric glove (or polymeric coating on a fabric liner of a supported glove) comprising a barrier coating, can be between about 10-20 mil (single-walled thickness) that provides protection against liquid permeability. Such gloves comprise, for example, NBR, NR, PI, CR, and PU, as discussed above, and further comprise a flow modifier (e.g., styrene-mono secondary butyl maleate-monomethyl maleate-maleic anhydride polymer), curative agents, germicide, pigments, and water. After forming the fluid repellent coating, a primer coagulant can be applied prior to applying an exterior coating. The primer coagulant's function is similar to a gelled coagulant but optional. A basic formulation comprises calcium nitrate, a wetting agent (e.g., alkyl trimethylammonium bromide), and water.

Embodiments according to the invention include the use of a dispersion that comprises microparticles, for example, silica particles functionalized with fluorine chemistry, for example, HeiQ® Barrier RCF, and a fluorocarbon to promote the uniform dispersion of the microparticles, such as HeiQ® Barrier HM (both obtained from HeiQ® Materials AG). HeiQ® Barrier HM is a liquid formulation containing fluorine resin chemistry and auxiliary components to promote uniform coverage on the treatment surface. HeiQ® Barrier RCF is a liquid formulation containing specially engineered silicon dioxide (silica) particles that are functionalized with fluorine chemistry in amounts effective to increase fluid repellency on the exterior surface relative to the same barrier having only the hydrophobic chemical (in similar amounts). After forming the barrier article (after forming an elastomeric glove or coated fabric glove, by, for example, a dipping process) or, alternatively, using a preformed barrier article, the article is dipped into a formulation (for example, an aqueous formulation) of the hydrophobic micro-particles and/or the hydrophobic chemical. For example, a useful combination of hydrophobic micro-particles and hydrophobic chemical is HeiQ® Barrier RCF (for example at 10-100 g/L), and HeiQ® Barrier HM (for example at 20-110 g/L). If separate formulations are used, the hydrophobic chemical formulation can usefully be dipped second. Also, because the formulations are suspensions, stirring during dipping can be helpful.

### Treatment Formulations

Various formulations of HeiQ® Barrier RCF and HeiQ® Barrier HM can be used for repellent treatments. HeiQ® Barrier RCF also can be used with other existing fluorine finishes. In this study, high, medium and low dosages of HeiQ® mixture were investigated. The combinations of these dosages are given in Table 1.

TABLE 1

| | Mixture Formulation | | | |
|---|---|---|---|---|
| | HeiQ ® | HeiQ ® | Acetic | |
| | Barrier | Barrier | acid | Water |
| | RCF (grams) | HM (grams) | 80% (ml) | (grams) |
| High dosage | 68 | 90 | 1 | 1000 |
| Medium dosage | 39 | 51 | 1 | 1000 |
| Low dosage | 27 | 36 | 1 | 1000 |

To confirm the advantages of HeiQ® treatment over conventional fluorine chemistry, a treatment solution solely based on fluorine was also studied. The repellent coating in this solution was Oleophobol® CP-SLA—an aqueous dispersion of perfluorinated acrylic copolymer supplied by Huntsman. Formulation of this fluorine treatment is given in Table 2. The formulations were applied as outlined in Table 7, which is discussed in detail below.

TABLE 2

| | Mixture Formulation | | | | |
|---|---|---|---|---|---|
| | | HeiQ ® | HeiQ ® | Acetic | |
| | Oleophobol ® | Barrier | Barrier | acid | Water |
| | CP-SLA (g) | HM (g) | RCF (g) | 80% (ml) | (g) |
| Conventional | 90 | | | 1 | 1000 |
| Fluorine | | | | | |
| treatment (CF) | | | | | |
| HeiQ ® | | 90 | 68 | 1 | 1000 |
| treatment (HQ) | | | | | |

### Tests

Various tests were performed on several gloves, including polyisoprene, natural rubber, and polychloroprene, according to embodiments of the invention as well as non-treated gloves for controls. Visual indicia of the efficacy of treatments according to the invention are shown and are labeled Experimental or Treated while prior art gloves are labeled Conventional or Control.

Visual repellency test with water: Glove according to embodiments of the invention and control gloves were mounted onto a former and immersed into an aqueous solution containing water and red pigment (Farsperse Red PR1123). Immediately after the withdrawal of each sample, the effect of water repellency on the glove surface was photographed.

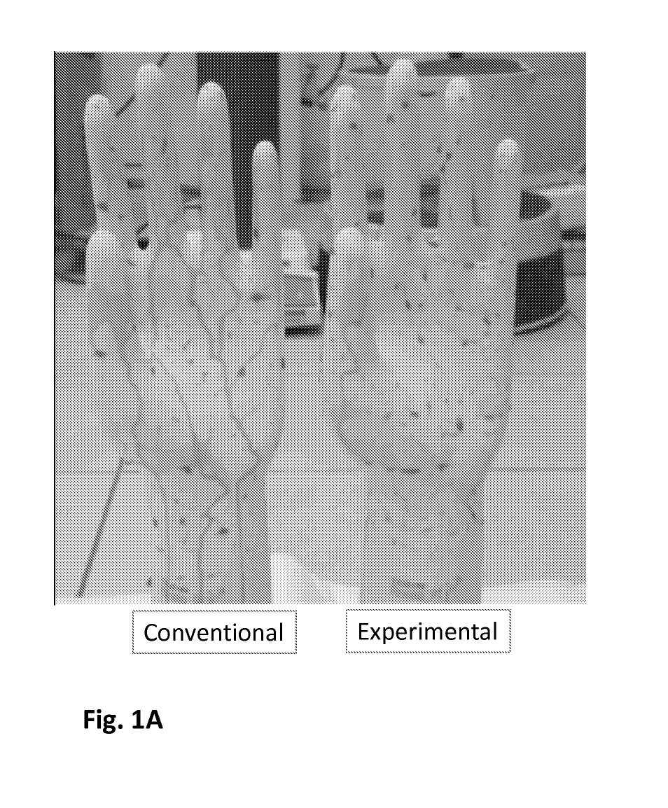

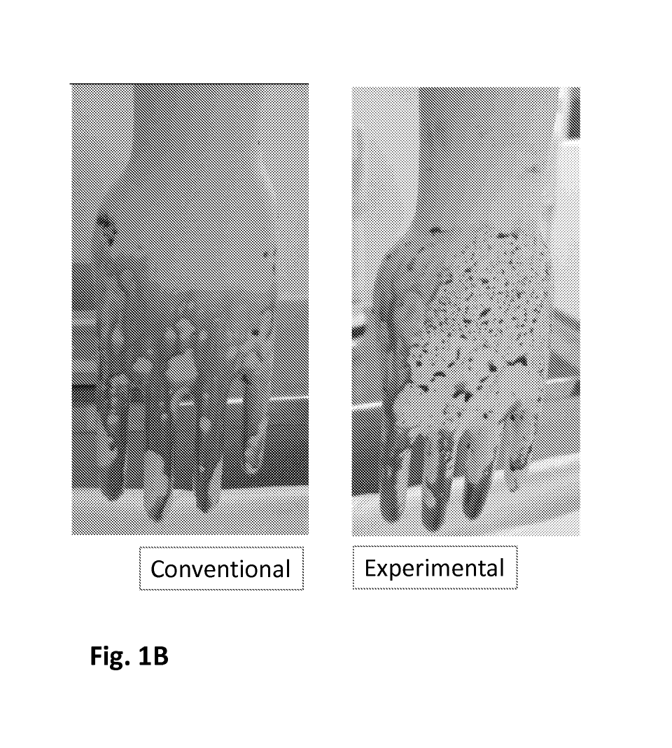

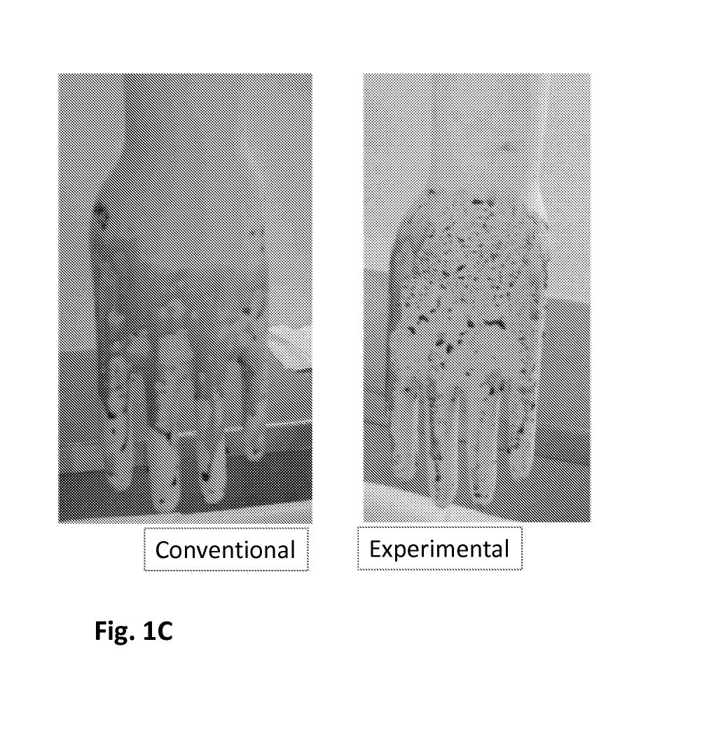

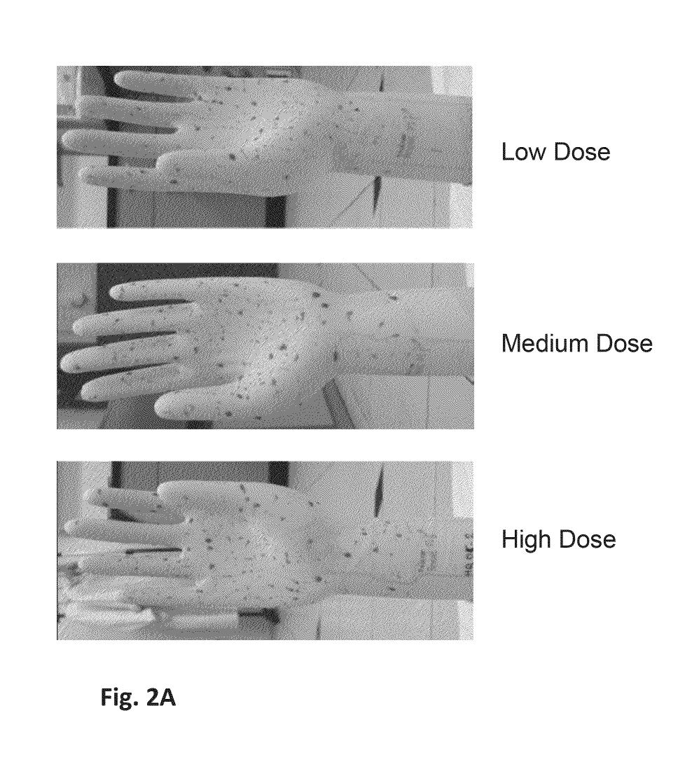

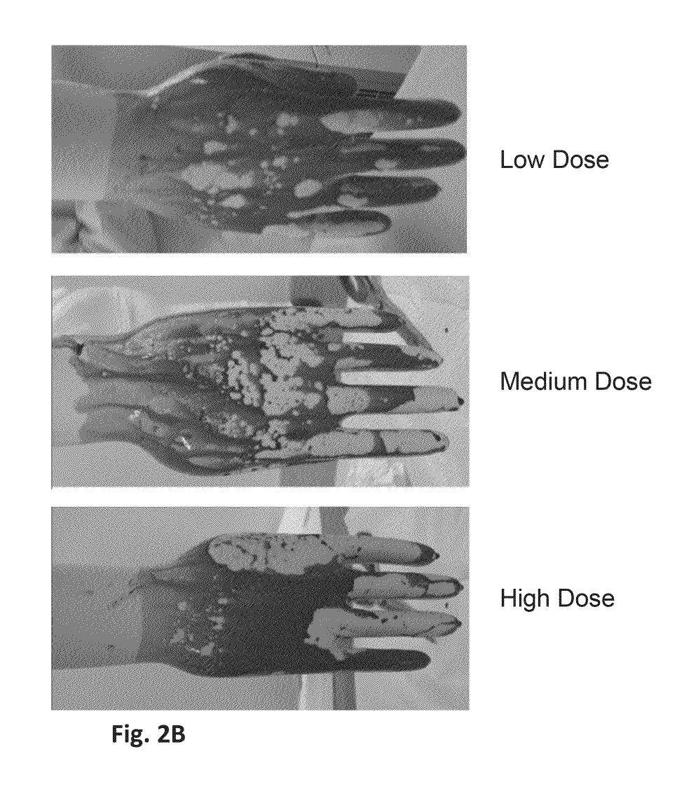

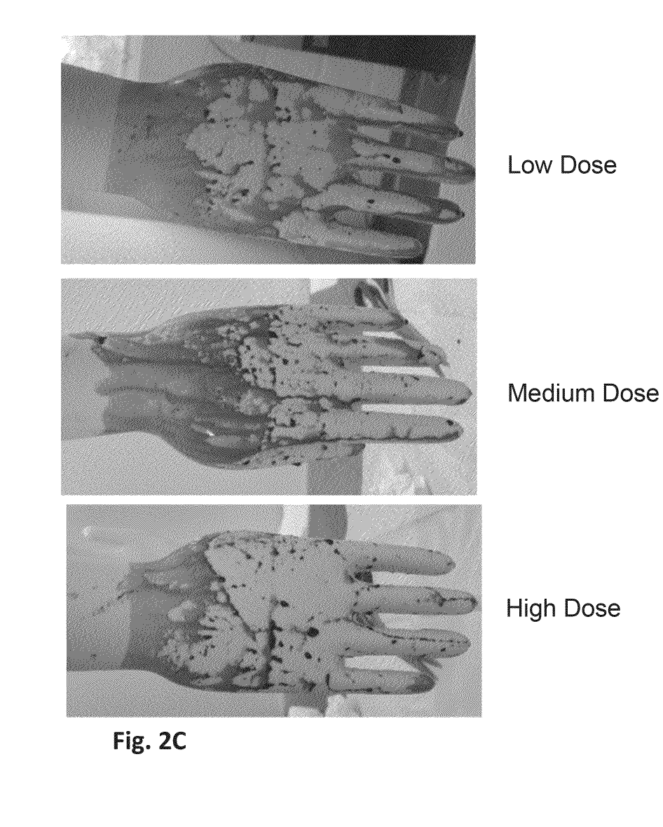

Visual repellency test with human blood: Expired human blood sample (Type O, 2 months expired) collected from National Blood Bank, Kuala Lumpur, Malaysia was used for the test. Treated and control gloves were mounted onto a handed former and immersed in human blood at approximately 20° C. and withdrawn. Immediately following withdrawal, the glove surface was photographed showing the effect of blood repellency on the glove surface. The glove surface was then photographed again after 5 minutes, as shown in FIGS. 1 and 2, respectively.

Contact angle test: Contact angle test with water was measured on VCA Optima Instrument. A 1″×1″ glove film specimen was cut and 7 microliter of distilled water (a droplet) was deposited onto its surface. A digital photograph was taken immediately following the deposition of the droplet and marked as initial contact angle measurement. Additional photographs of the droplet were taken at following 5, 15, 20, 40, and 60 minutes intervals after deposition. Contact angle retention (%) at certain interval time was calculated by the ratio of contact angle at that particular time and the initial contact angle.

Tensile properties test: Tensile properties of control and treated gloves were tested according to ASTM D412. Tensometer Monsanto T10 was used to conduct the tensile test and dumbbell die cutter C was used. The accelerated aging of test specimens was carried out in accordance with ASTM D537-04. Irradiation created by Cobalt-60 Gamma ray source minimum dose of 2.5 mrads was used to sterilize the glove samples.

Conventional Vs. Combination Treatment

The treatment combination in accordance with embodiments of the invention was evaluated in comparison with conventional fluorine treatment formulation (Conventional). The treatment was done on PI gloves and mixture formulations are given in Table 2. Gloves were mounted onto the former which was approximately ½ size smaller than the glove. The Glove and former were then dipped into the treatment solution with the following dipping profile (immerse-dwell-withdraw) 10-10-10 seconds.

FIG. 1A shows a visual water repellency test for PI gloves made more repellent with a conventional fluorocarbon treatment, or with a treatment according to embodiments of the present invention. FIG. 1A shows the water repellency of a glove treated in accordance with embodiments of the invention compared with that of a conventional fluorine treatment glove. It is observed that both treatment methods significantly improve water repellency. However, the treatment of the present invention provided higher repellency level as the amount of water picked up was less on the surface of the HeiQ® treated glove.

FIGS. 1B and 1C show a visual blood repellency test for PI gloves made more repellent with a conventional fluorocarbon treatment, or with a treatment according to embodiments of the present invention, the gloves shown immediately after dipping in blood, and 5 minutes thereafter, respectively. FIGS. 1B and 1C show the visual repellency test of conventional gloves and gloves in accordance with embodiments of the present invention with human blood. It is clearly seen that blood repellency of HeiQ® glove is better than that of conventional fluorine treatment glove. The amount of blood being picked up was less and also less stain remained on the surface of the HeiQ® treated glove.

The above results confirm that gloves treated according to embodiments of the invention, and therefore with the incorporation of microstructure particles into fluorine, significantly improve water and blood repellency of PI surgical glove compared with that of the conventional fluorine treatment. Therefore, PI gloves comprising a medium to medium-high dosage of HeiQ® RCF and HM components provide unexpectedly increased blood repellency. At least one embodiment according to the invention comprises treating a PI glove with a composition including approximately 50 grams HeiQ® RCF, 70 grams of HeiQ® HM, one gram of acetic acid in 1000 grams of water. PI gloves at this range of dosage showed a particularly surprising liquid repellency. Further tests were carried out to investigate the effect of different dosages of HeiQ® treatment on water and other liquids repellency.

FIG. 2A shows a visual water repellency test for PI gloves having an increased repellency effect with different amounts of the treatment of the invention. FIG. 2A shows photographs of PI glove surface treated with high, medium, and low dosages (Table 1) after immersion and withdrawal from the water pigment solution. It can be seen that the water beads are formed on surface of all gloves.

The contact angle of water droplets on the surfaces of gloves treated at various dosages and retention ratio after 60 minute interval are shown in Table 3. Similar with the visual test result, the difference in contact angle and retention ratio among different gloves were not significant.

TABLE 3

| | Contact angle (degree) after interval time | | | | | | Retention |
|---|---|---|---|---|---|---|---|
| Dosage | | | 15 | 20 | 40 | | ratio after |
| of HeiQ® | 0 min | 5 min | min | min | min | 60 min | 60 min (%) |
| High | 120.5 | 109.7 | 106.7 | 105.8 | 100.9 | 93.3 | 77 |
| Medium | 116.7 | 114.9 | 109.1 | 107.8 | 102.4 | 93.8 | 80 |
| Low | 116.6 | 114.3 | 113.1 | 109.0 | 100.7 | 93.4 | 80 |

FIGS. 2B and 2C show a visual blood repellency test for PI gloves made more repellent with different amounts of a treatment according to embodiments of the present invention. FIGS. 2B and 2C show photographs of PI glove surface treated with high, medium and low dosages (Table 1) after immersion and withdrawal from human blood, initially (FIG. 2B), and after 5 minutes (FIG. 2C).

As can be observed from FIGS. 1A-1C and FIGS. 2A-2C, the treatments in accordance with the invention improve blood repellency of the glove surface. By this measure, the higher the dosage, the better the repellency effect.

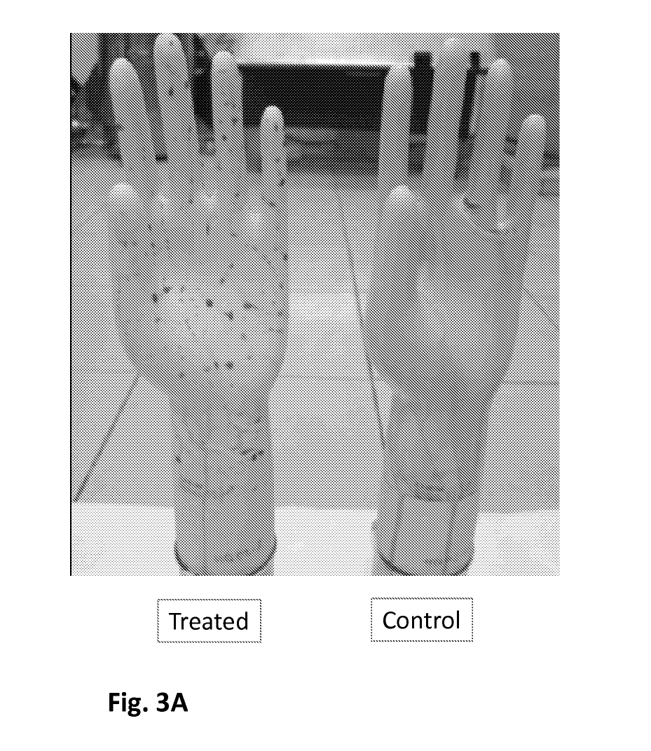

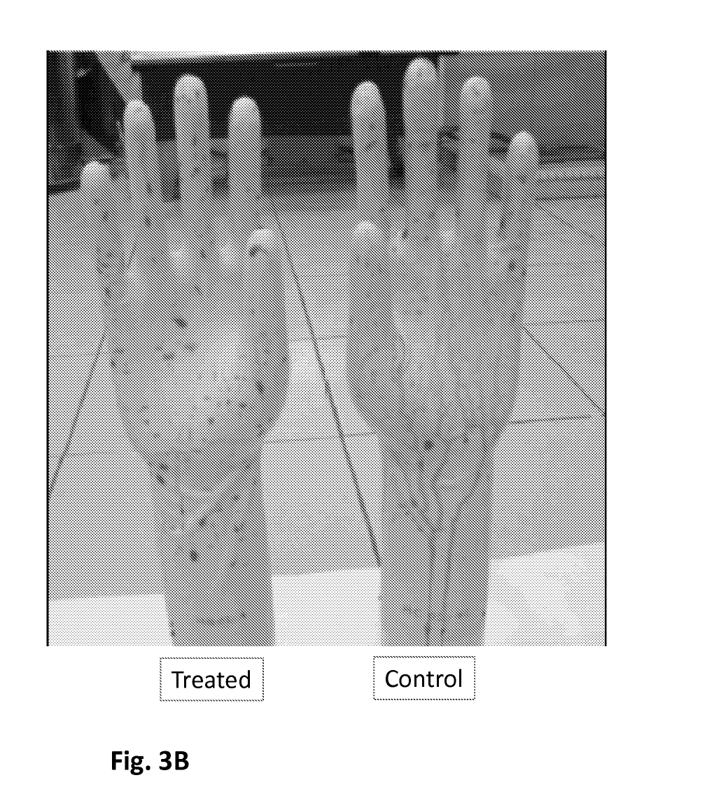

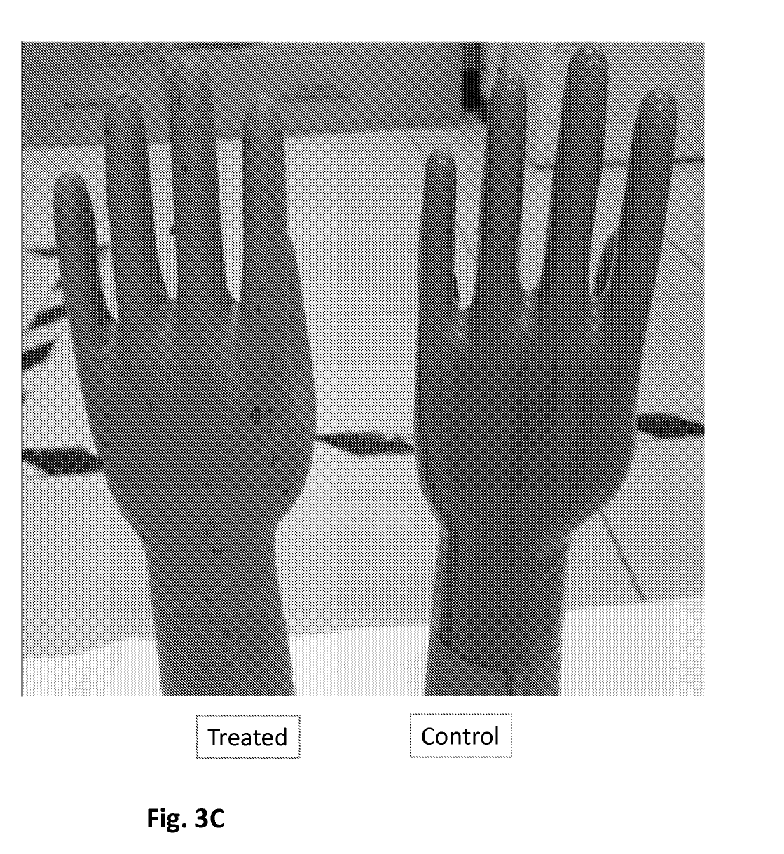

High dosage treatment was tested on other types of gloves, namely NR and CR gloves, with comparison to PI gloves. FIGS. 3A, 3B, and 3C show a visual water repellency test for PI gloves, NR gloves, and CR gloves. The gloves treated according to embodiments of the invention are on the left sides of the figures, and the non-treated controls on the right. FIGS. 3A-3C show photographs of different gloves treated with high dosage immediately after withdrawal from pigment water. It can be seen that high dosage HeiQ® treatment significantly improved water repellency on the surface of all treated gloves. The level of improvement is more prominent toward CR gloves. For NR gloves, the control glove showed some level of water repellency, which may be due, without intending to be bound by theory, to a texture on the palm area, which created a rougher surface similar to the particle deposition of the glove treatments according to embodiments of the invention.

Contact angle of water droplets deposited on the surface of various types of gloves are presented in Table 4. The initial contact angle of gloves treated with the high dosage in Table 1 was greater than 115° and retention ratios after a 60 minute interval were greater than 70%.

TABLE 4

| | Contact angle (degree) after interval time | | | | | | Retention |
|---|---|---|---|---|---|---|---|
| Type of | | | 15 | 20 | 40 | | ratio after |
| glove | 0 min | 5 min | min | min | min | 60 min | 60 min (%) |
| PI Control | 75.1 | 71.5 | 65.3 | 62.4 | 53.3 | 42.5 | 57 |
| PI Treated | 120.5 | 109.7 | 106.7 | 105.8 | 100.9 | 93.3 | 77 |
| NR Control | 129.4 | 112.5 | 106.4 | 104.5 | 97.6 | 81.1 | 63 |
| Front* | | | | | | | |
| NR Control | 92.2 | 83.8 | 79.3 | 74.2 | 63.5 | 48.2 | 52 |
| Back | | | | | | | |
| NR Treated | 125.5 | 114.7 | 108.6 | 104.1 | 96.9 | 85.8 | 68 |
| Front | | | | | | | |
| NR Treated | 124.1 | 103.6 | 101.7 | 99.9 | 94.7 | 87.8 | 71 |
| Back | | | | | | | |
| CR Control | 67.5 | 64.4 | 58.1 | 56.1 | 45.0 | 35.6 | 53 |
| CR Treated | 115.3 | 105.5 | 103.3 | 102.4 | 92.6 | 82.9 | 72 |

For NR gloves, consistent with visual repellency test results, the contact angles and retention ratio of the control glove were relatively high on the front side (textured) compared with that on the back side. However, after treatment, both sides possessed quite similar contact angle values.

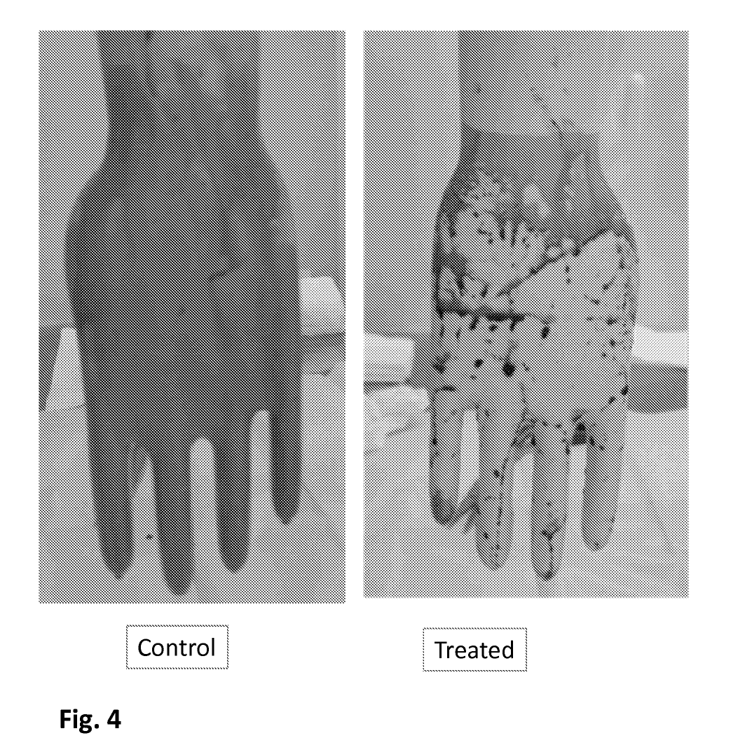

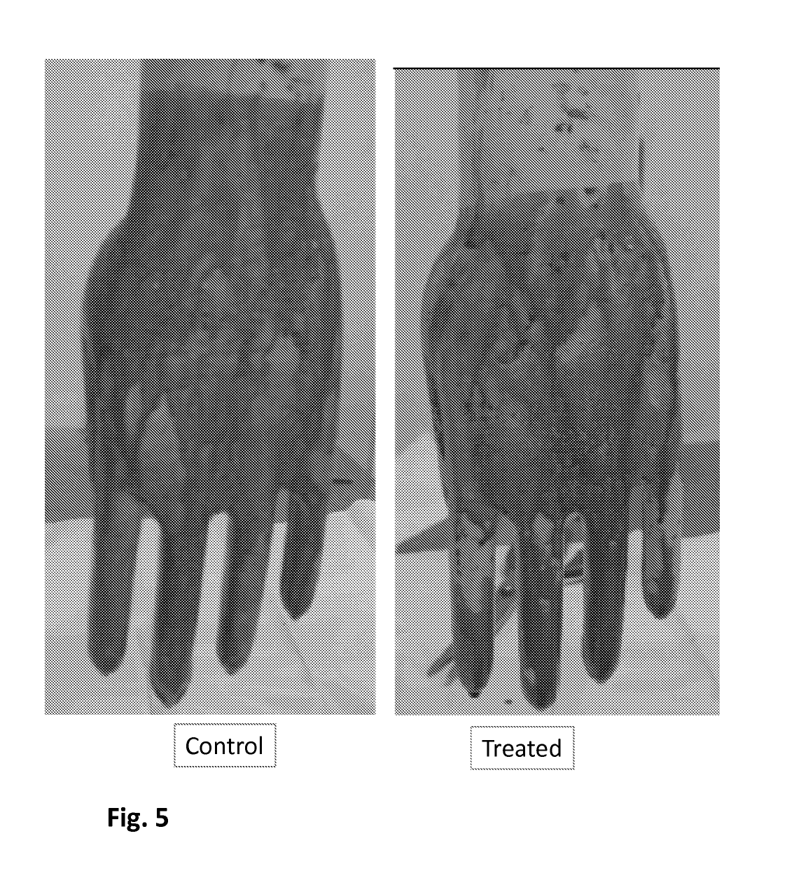

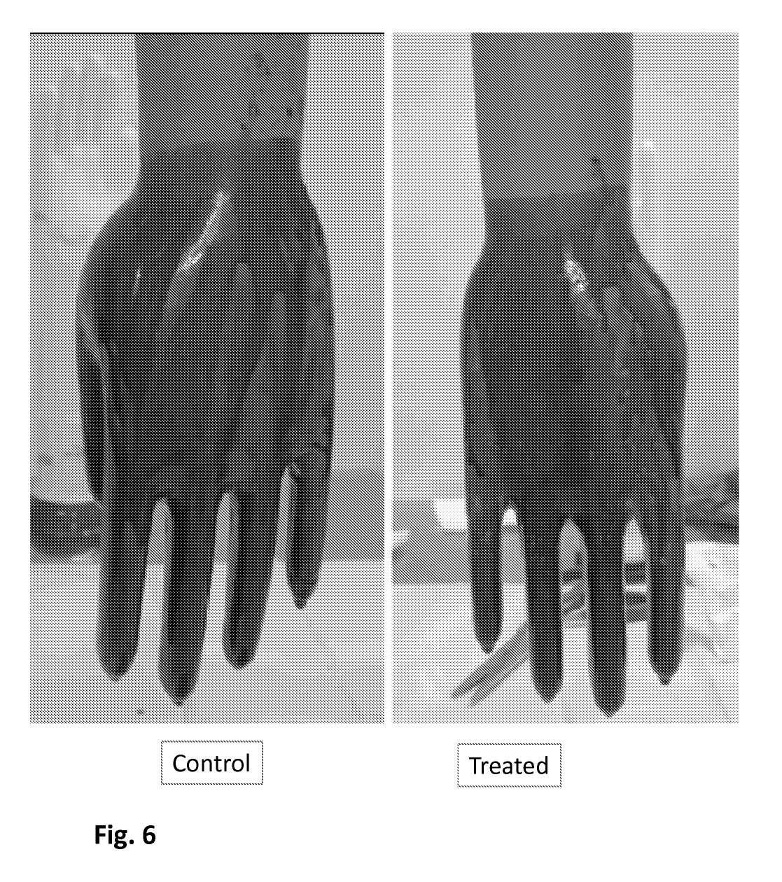

FIGS. 4-6 show a visual blood repellency test for PI, NR, and CR gloves made more repellent with embodiments according to the treatment of the invention (high dose), the gloves shown 5 minutes thereafter dipping in blood. FIG. 4 shows photographs taken after withdrawal from the blood for PI gloves. FIG. 5 shows photographs taken 5 minutes after withdrawal from the blood for NR gloves. FIG. 6 shows photographs taken 5 minutes after withdrawal from the blood for polychloroprene gloves. The repellency toward human blood was most clearly observed on the surface of the PI gloves.

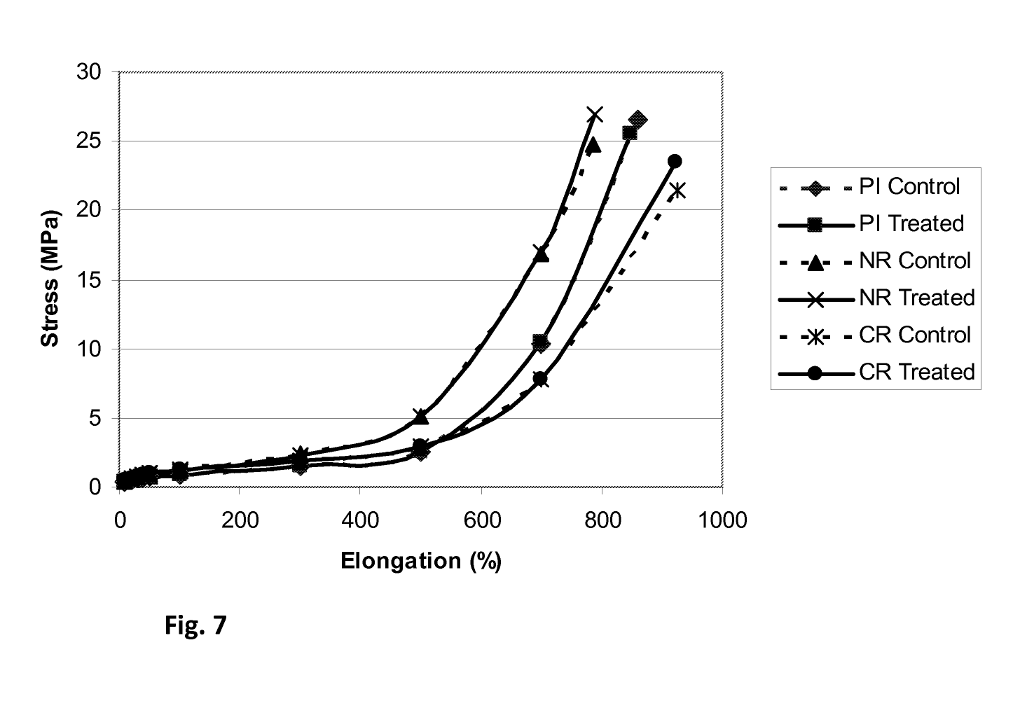

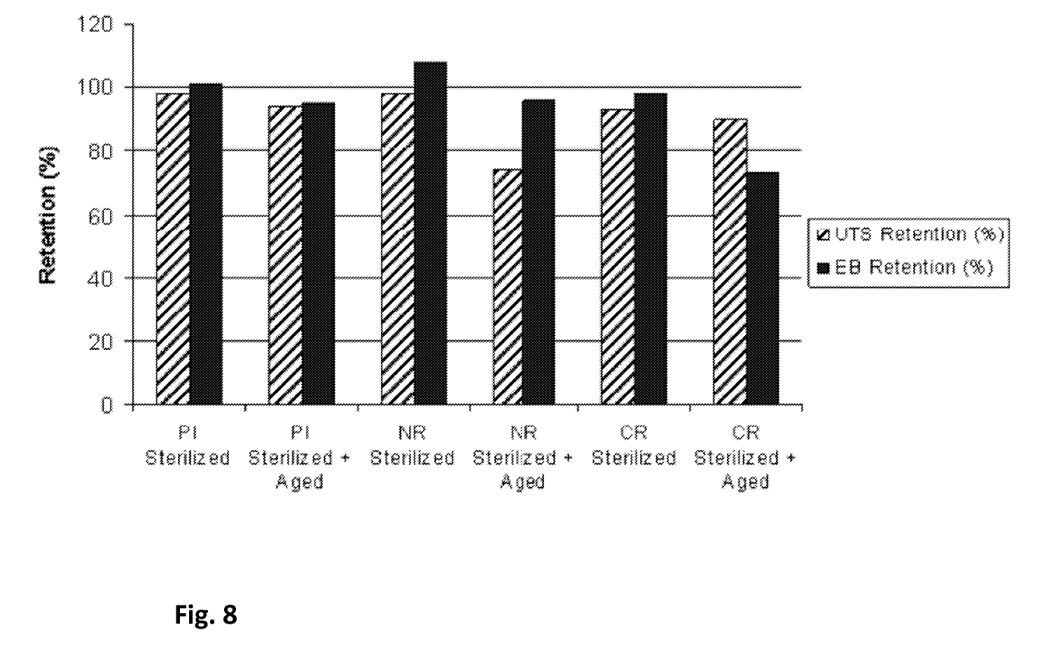

FIG. 7 shows the stress-strain curves for PI, NR, and CR gloves that were treated or un-treated (control) with embodiments according to the treatment of the invention. It can be seen that the stress-strain behavior of the treated gloves was quite similar with that of control gloves. FIG. 8 shows the ultimate tensile strength (UTS) and elongation of break of PI, NR, and CR gloves that are sterilized, or sterilized and aged, measured (by %) relative to the treated glove without sterilization or aging.

Table 5 shows the ultimate tensile strength and elongation at break of various gloves treated with high dosage, before and after sterilization and aging. It can be observed that the physical properties of all gloves still met the requirements of ASTM D 3577, the Standard Specification for Rubber Surgical Gloves.

TABLE 5

| Type of glove | UTS (MPa) | EB (%) |
|---|---|---|
| PI Treated | 25.57 | 848 |
| PI Treated Sterilized | 25.10 | 853 |
| PI Treated Sterilized + Aged | 23.68 | 808 |
| NR Treated | 26.97 | 789 |
| NR Treated Sterilized | 26.34 | 854 |
| NR Treated Sterilized + Aged | 19.53 | 816 |
| CR Treated | 23.47 | 924 |
| CR Treated Sterilized | 21.71 | 909 |
| CR Treated Sterilized + Aged | 19.65 | 660 |

Compared with control gloves, as shown in FIG. 8, the retention of ultimate tensile strength and elongation at break after sterilization and aging of different treated gloves was high, i.e., no significant degradation on physical properties of the treated gloves when subjected to stringent conditions of manufacturing processes of surgical gloves. The process to form gloves, according to embodiments of the invention, with optional steps, includes the steps in Tables 6 and 7.

TABLE 6

| Step | Purpose | Action | |
|---|---|---|---|
| 1 | Glove preparation | Provide dry gloves that are substantially free of residual chemicals | |
| 2 | Recipe preparation | Prepare formulation ingredients for a batch | |
| 3 | Prepare dispersion | Prepare dispersion having microparticles | |
| 4 | Preparation of | 1. | Stir in a fluorocarbon dispersion and microparticles |
| | examples for a final | | dispersion in a container for approximately 1 minute, or in |
| | batch formulation | | some embodiments, approximately, several minutes |
| | | 2. | Deliver water to approx. 50% of required amount |
| | | 3. | Add Acetic Acid into the container while stirring slowly |
| | | 4. | Add microparticles dispersion while stirring container |
| | | 5. | Add fluorocarbon dispersion while stirring container |
| | | 6. | Deliver remainder of water to 100% of required amount |
| | | 7. | Stir |

| | | |
|---|---|---|
| 5 | Transferring to | Transfer batch formulation to a dipping tank |
| | dipping tank | |
| 6 | Dipping | Mount glove on a former, approx. ½ size smaller (if not already |
| | | mounted, as on a former) Immerse glove into the mixture, dwell, and |
| | | withdraw. Allow gloves to drip dry for approx. 3 minutes. |
| 7 | Drying | Dry at approx. 100-120° C. for ~7 min. or until glove surface dry |
| 8 | Curing | Cure dry gloves at approx. 150-160° C. for approx. 1-2 minutes |

TABLE 7
Exemplary Siliconizing Formulation

| | Ingredient | Unit | Batch |
|---|---|---|---|
| 1 | Water | ml | 781.8 |
| 2 | dimethicone emulsion | g | 8 |
| 3 | cationic emulsion of an amine-functional silicone polymer | g | 1.03 |
| 4 | nonionic polypropylene emulsion | g | 9.2 |
| | TOTAL WEIGHT | g | 800 |

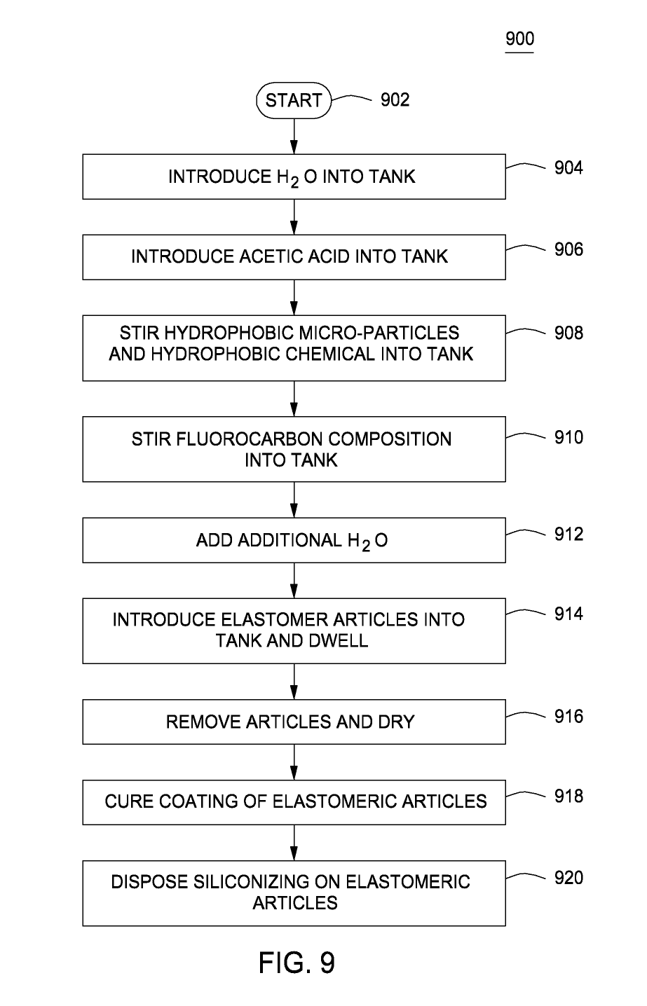

FIG. 9 depicts a flow diagram 900 to surface treat an article having an elastomeric surface, according to embodiments of the invention. The method 900 starts at step 902 and proceeds to step 904, at which point water is introduced to a container. The method 900 proceeds to step 906, where acetic acid is introduced into the container. At step 908, a combination of hydrophobic micro-particles and hydrophobic chemical are stirred into the container containing the water and acetic acid. At step 910, a fluorocarbon composition is stirred into the container, followed by a second amount of water being stirred into the container at step 912 to form the treatment formulation. Next, at step 914, articles, such as a supported or unsupported glove having an elastomeric surface, are introduced into the container and allowed to dwell within the container. At step 916, the articles are removed and allowed to drip dry, and/or dried at, for example, 110-120° C. for 5-10 minutes. At step 918, the articles having a surface treated coating are cured at approximately 150-160° C. for approximately 1-2 minutes. At step 920, the articles are optionally immersed in a siliconizing formulation, e.g., the siliconizing formulation described above in Table 7, to siliconize the articles. Step 920 comprises, for example, tumbling the articles in a drum for, for example 3-10 minutes, stopping the tumbling for, for example, five minutes, and repeating for one or more cycles.

The articles may then be optionally washed in water and dried, for example, at approximately 68-76° C. for several minutes. The siliconizing process, as discussed above in step 920, imparts lubricious properties allowing the ease of donning and doffing article, especially, for example, gloves. Any article, including gloves, are placed into a drum and tumbled with a siliconizing formulation. The formulation comprises a dimethicone emulsion, a cationic emulsion of an amine-functional silicone polymer, and a nonionic polypropylene emulsion. In some embodiments in accordance with the invention, the dimethicone emulsion is Dow Corning 365, the cationic emulsion of an amine-functional silicone polymer is Dow Corning 939, and the nonionic polypropylene emulsion is Michem® Emulsion 43040. In some embodiments, the formulation is the exemplary formulation as in Table 7.

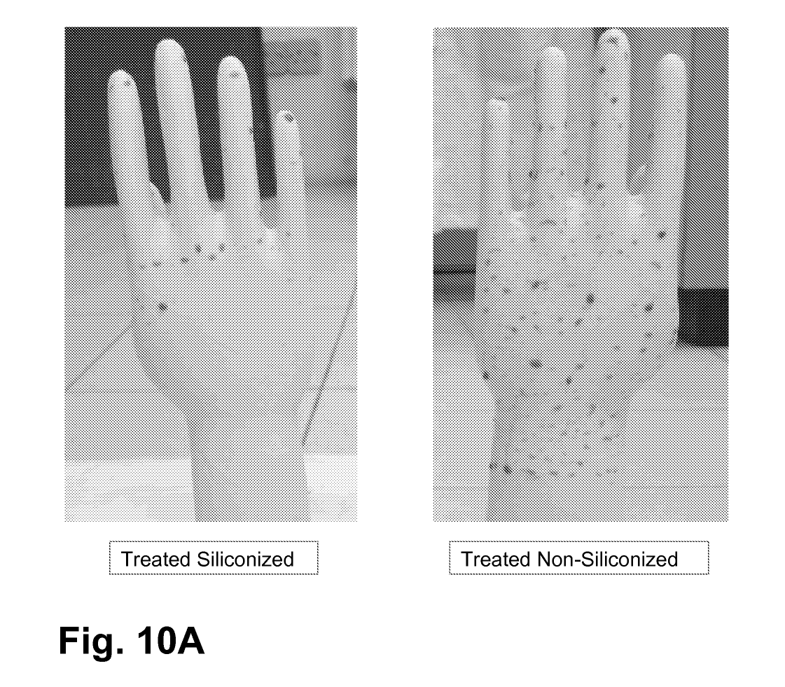

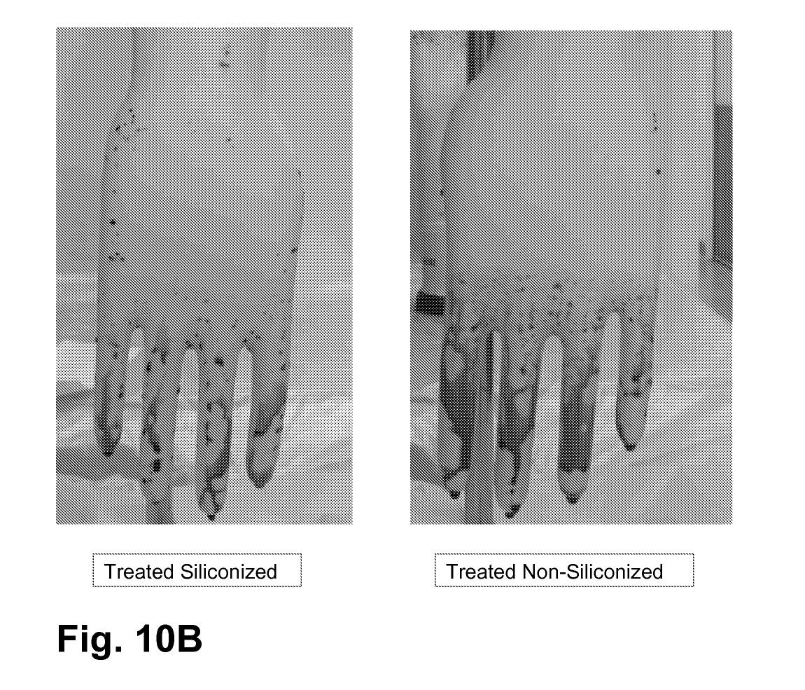

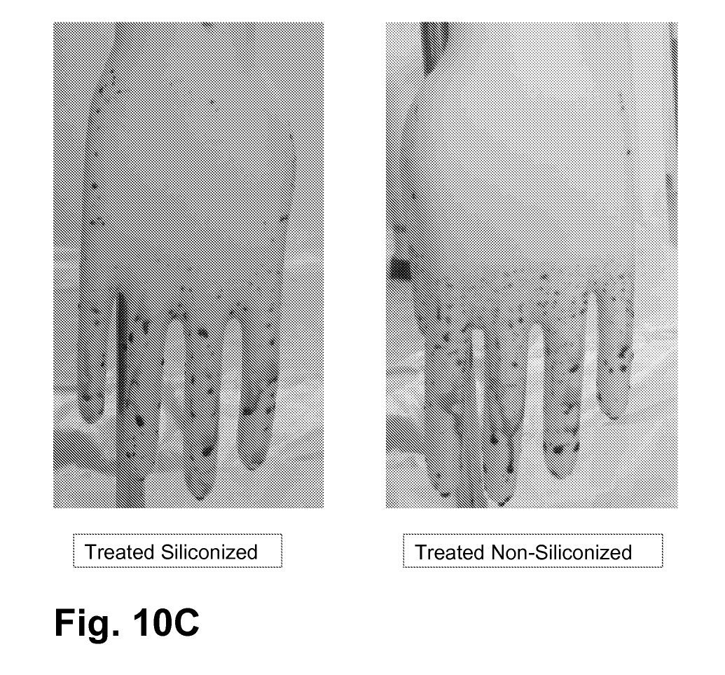

To evaluate the enhancement of the siliconizing process on blood repellency; treated siliconized and treated non-siliconized gloves were mounted onto a former and immersed in human blood at approximately 20° C. for 15 minutes. FIG. 10A shows a visual water repellency test for treated siliconized and treated non-siliconized PI gloves. FIGS. 10B and 10C show a visual blood repellency test for treated siliconized and treated non-siliconized PI gloves. Immediately following withdrawal, the glove surface was photographed showing the effect of blood repellency on the glove surface. The glove surface was then photographed again after an additional 1 minute, as shown in FIGS. 10B and 10C, respectively.

It is observed that siliconizing significantly enhances both water and blood repellency of treated gloves to an unexpected degree. FIG. 10A shows the water repellency of a treated siliconized glove compared with that of a treated non-siliconized glove. It is clearly seen that only a few water droplets remain attached to surface of the treated siliconized glove whereas a lot of water droplets remain on the surface of the treated non-siliconized glove. It is observed that blood repellency is also significantly improved with siliconizing as shown in FIGS. 10B-10C. Therefore, although the combination of hydrophobic micro-particles and a hydrophobic fluorocarbon on a polyisoprene elastomeric surface, as described above in connection with, for example, FIGS. 1-4 produces an enhanced water and/or blood repellent surface, the disposition of the siliconizing formulation comprising the dimethicone emulsion, the cationic emulsion of an amine-functional silicone polymer, and the nonionic polypropylene emulsion on the hydrophobic micro-particles and hydrophobic fluorocarbon treated glove results in an even greater and unexpectedly enhanced water and/or blood repellent surface.

In some embodiments of the invention, a coagulant solution (e.g., an aqueous solution containing a salt, e.g., 1-20 wt % calcium nitrate, calcium chloride, calcium citrate, and the like) is applied to a former, a former dressed with a fabric liner, or an elastomer layer and dried prior to application of a subsequent elastomer layer. In some embodiments, the former is not treated with coagulant prior to dipping a rough-surfaced former into foamed elastomer. In certain embodiments, the liquid resistant layer of elastomer is not treated with coagulant prior to dipping into foamed elastomer. In certain embodiments, the liquid resistant layer of elastomer is leached with water (such as, for example, 40° C. water) prior to dipping into foamed elastomer.

When applying a polymeric layer to a former, a gelled coagulant can be used to promote adhesion of the polymeric, elastomeric, or latex composition to adhere to the former. The polymeric, elastomeric, or latex composition comprises, for example, calcium nitrate, a wetting agent (alcohol ethoxylate and/or alkylarylalkoxylate), a cellulosic thickener, a water-based defoamer, which is a bubble inhibitor, and water. The formulation can be designed to have few wetting agents in order to have optimum former wetness to minimize potential of major defects such as holes and thin spots.

All ranges recited herein include ranges therebetween, inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4 . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent ranges starting at integer values at the recited order of magnitude or one lower, e.g., 3 or more includes 4 or more, or 3.1 or more.

The foregoing description of embodiments of the invention comprises a elements, devices, machines, components and/or assemblies that perform various functions as described. These elements, devices, machines, components and/or assemblies are exemplary implementations of means for performing their functions.

Although only a few exemplary embodiments of the present invention have been described in detail above, those skilled in the art will readily appreciate that many modifications are possible in the exemplary embodiments without materially departing from the novel teachings and advantages of this invention.

What is claimed is:

1. A method for creating a fluid repellent article having an elastomeric surface, comprising:
combining hydrophobic micro-particles and a hydrophobic fluorocarbon, water, and acetic acid to form a mixture;
immersing an article having an elastomeric surface into the mixture;
drying the elastomeric surface; and
immersing the article having the elastomeric surface in a siliconizing formulation to siliconize the elastomeric surface.

2. The method of claim 1, wherein the hydrophobic micro-particles is a dispersion of HeiQ® RCF.

3. The method of claim 1, wherein the hydrophobic fluorocarbon is HeiQ® HM.

4. The method of claim 1, wherein the siliconizing formulation comprises an aqueous solution of a dimethicone emulsion, a cationic emulsion of an amine-functional silicone polymer, and a nonionic polypropylene emulsion.

5. The method of claim 4, wherein the dimethicone emulsion, the cationic emulsion of an amine-functional silicone polymer, and the nonionic polypropylene emulsion are Dow Corning 365, Dow Corning 939, and Michem® Emulsion 43040 respectively.

6. The method of claim 4, wherein the siliconizing formulation comprises approximately 1% Dow Corning 365, 0.125% Dow Corning 939, and 1.125% Michem® Emulsion 43040.

7. The method of claim 4, wherein the elastomeric surface is immersed in the HeiQ® Barrier RCF and HeiQ® Barrier HM mixture for approximately 1-3 minutes.

8. The method of claim 1, wherein the elastomeric surface is mounted on a former before the immersing step.

9. The method of claim 1, wherein the elastomeric surface is allowed to drip dry for 1-5 minutes.

10. The method of claim 1, wherein the elastomeric surface is dried at a temperature of approximately 100-130° C.

11. The method of claim 1, wherein the elastomeric surface is cured at a temperature ranging from approximately 140-180° C.

12. A fluid repellent glove, comprising:
a glove having an elastomeric surface;
a fluid repellent coating, comprising a hydrophobic chemical containing hydrophobic micro-particles and a fluorocarbon chemical disposed on the elastomeric surface; and
a silicon treatment disposed on the fluid repellent coating.

13. The fluid repellent glove of claim 12, wherein the elastomeric surface comprises natural rubber, polychloroprene, acrylonitrile butadiene copolymer, carboxylated acrylonitrile butadiene copolymer, synthetic polyisoprene, polyurethane, styrene-butadiene, butyl rubber, isobutylene and isoprene copolymer, or blends thereof.

14. The fluid repellent glove of claim 12 further comprising a fabric liner adhered to an interior surface of the elastomeric glove.

15. The fluid repellent glove of claim 12, wherein the fluid repellent coating comprises HeiQ® HM and HeiQ® RCF.

16. The fluid repellent glove of claim 12, wherein the silicon treatment comprises Dow Corning 365, Dow Corning 939, and Michem® Emulsion 43040.

17. The fluid repellent glove of claim 12, wherein the fluid repellent coating comprises a ratio dosage of a composition including approximately 50 grams HeiQ® RCF, 70 grams of HeiQ® HM, and 1 gram of acetic acid in 1000 grams of water.

18. The fluid repellent glove of claim 12, wherein the hydrophobic micro-particles are silica particles.

19. The fluid repellent glove of claim 12, wherein the micro-particles include nanoparticles, ranging in size from approximately 0.01 to 10 micrometers.

20. The fluid repellent glove of claim 12, wherein the surface treated glove induces a lotus effect with fluids.
